(19) United States
(12) Patent Application Publication
(10) Pub. No.: US 2023/0191403 A1
(43) Pub. Date: Jun. 22, 2023

(54) REPLACEMENT BOX FOR URINALYSIS DEVICE, REPLACEMENT BOX BOTTOM SUPPORT, AND REPLACEMENT BOX MOUNTING STRUCTURE

(71) Applicant: Shanghai Kohler Electronics, Ltd., Shanghai (CN)

(72) Inventor: Zhenwei Wang, Shanghai (CN)

(21) Appl. No.: 18/077,626

(22) Filed: Dec. 8, 2022

(30) Foreign Application Priority Data
Dec. 17, 2021 (CN) 202123196255.4

(51) Int. Cl. B01L 3/00

(52) U.S. Cl. CPC B01L 3/502715; B01L 2200/04; B01L 2200/025

(57) ABSTRACT

A replacement box for a urinalysis device includes a box body upper cover; a box body lower cover configured to be connected to the box body upper cover to form a box body cavity; and a first magnetic attraction device disposed at a bottom portion of the box body lower cover. The first magnetic attraction device is configured to guide the replacement box to reach a preset position. A replacement box bottom support for the urinalysis device includes: a bottom support body; a bottom support cavity disposed in the bottom support body and configured to receive a replacement box; and a second magnetic attraction device disposed at a bottom portion of the bottom support cavity. The second magnetic attraction device is configured to guide the replacement box to reach the preset position.

### CROSS REFERENCE TO RELATED APPLICATION

This application claims the benefit of priority to: Chinese Patent Application No. 202123196255.4 filed in the Chinese Intellectual Property Office on Dec. 17, 2021, which is hereby incorporated by reference in its entirety.

### FIELD

The present disclosure relates to the field of technologies related to a sanitary equipment, in particular to a replacement box for a urinalysis device, a replacement box bottom support, and a replacement box mounting structure.

### BACKGROUND

A urinalysis device may automatically use test paper for physical fitness testing. However, the test paper needs to be replaced by a customer after the test paper is used up.

However, when the customer manually mounts a replacement, it is difficult to detect whether the replacement is mounted or installed in place only by feeling or intuition. When the replacement is not mounted or installed in place, it is easy to cause a system action error.

Therefore, a position switch or an optocoupler switch is used to detect whether the replacement is in place. However, this detection method has a larger stroke and a lower stroke precision.

### SUMMARY

In view of the above technical problem, it is necessary to provide a replacement box for a urinalysis device, a replacement box bottom support, and a replacement box mounting structure to address the technical problem that the urinalysis device cannot effectively ensure in-place mounting or installation of a replacement for a urinalysis device.

The present disclosure provides a replacement box for a urinalysis device. The replacement box for a urinalysis device comprises a box body upper cover and a box body lower cover. The box body upper cover and the box body lower cover are connected to form a box body cavity, test paper is contained or housed in the box body cavity, and a bottom portion of the box body lower cover is provided with a first magnetic attraction device.

In an embodiment, the first magnetic attraction device is a magnet or a magnetic attraction iron sheet.

In an embodiment, an upper cover bottom surface of the box body upper cover protrudes beyond the box body lower cover.

The present disclosure further provides a replacement box bottom support for a urinalysis device. The replacement box bottom support comprises a bottom support body. A bottom support cavity for containing or receiving a replacement box is arranged in the bottom support body, a bottom portion of the bottom support cavity is provided with a second magnetic attraction device, and a detection switch is also fixed on the bottom support body.

In an embodiment, the second magnetic attraction device is a magnetic attraction iron sheet or a magnet.

In an embodiment, the replacement box bottom support further comprises an indicating device communicably coupled to the detection switch.

The present disclosure further provides a replacement box mounting structure for a urinalysis device. The replacement box mounting structure comprises the replacement box as described above and the replacement box bottom support as described above. The replacement box is inserted into the replacement box bottom support along a first direction, and the first magnetic attraction device and the second magnetic attraction device are at least partially overlapped along the first direction.

In an embodiment, the first magnetic attraction device is a magnet and the second magnetic attraction device is a magnetic attraction iron sheet. Alternatively, the first magnetic attraction device is a magnetic attraction iron sheet and the second magnetic attraction device is a magnet.

In an embodiment, the box body lower cover is inserted into the bottom support cavity, and the box body upper cover presses the detection switch.

In an embodiment, a height from the upper cover bottom surface of the box body upper cover protruding beyond the box body lower cover to a bottom portion of the first magnetic attraction device is smaller than a height from a top portion of the second magnetic attraction device to a top portion of the detection switch.

According to the present disclosure, the replacement box is guided and positioned to a preset position through the first magnetic attraction device and the second magnetic attraction device, and detection is conducted through the detection switch. Thus, a guiding effect is provided for a user to mount the replacement box, and an in-place detection precision is improved.

### BRIEF DESCRIPTION OF THE FIGURES

FIG. 1 is a schematic structural diagram of a replacement box for a urinalysis device according to an example of the present disclosure;

FIG. 2 is a schematic structural diagram of a replacement box bottom support for the urinalysis device according to an example of the present disclosure;

FIG. 3 is a top view of the replacement box bottom support for the urinalysis device according to an example of the present disclosure;

FIG. 4 is a sectional view of a replacement box mounting structure for the urinalysis device according to an example of the present disclosure; and

FIG. 5 is a flow chart of a method for assembling the replacement box mounting structure for the urinalysis device according to an example of the present disclosure.

### DESCRIPTIONS OF REFERENCE NUMERALS

1 refers to a replacement box; 11 refers to a box body upper cover; 111 refers to an upper cover bottom surface; 12 refers to a box body lower cover; 13 refers to a box body cavity; 14 refers to test paper; 15 refers to a first magnetic attraction device; 2 refers to a replacement box bottom support; 21 refers to a bottom support body; 22 refers to a bottom support cavity; 23 refers to a second magnetic attraction device; and 24 refers to a detection switch.

### DETAILED DESCRIPTION

Specific embodiments of the present disclosure are further described hereinafter with reference to the drawings. The same or equivalent parts are denoted by the same reference numerals. It should be noted that the terms “front”, “back”, “left”, “right”, “up” and “down” used in the following description refer to the directions in the drawings, and the terms “inner” and “outer” refer to the directions towards or far away from geometric centers of specific parts respectively.

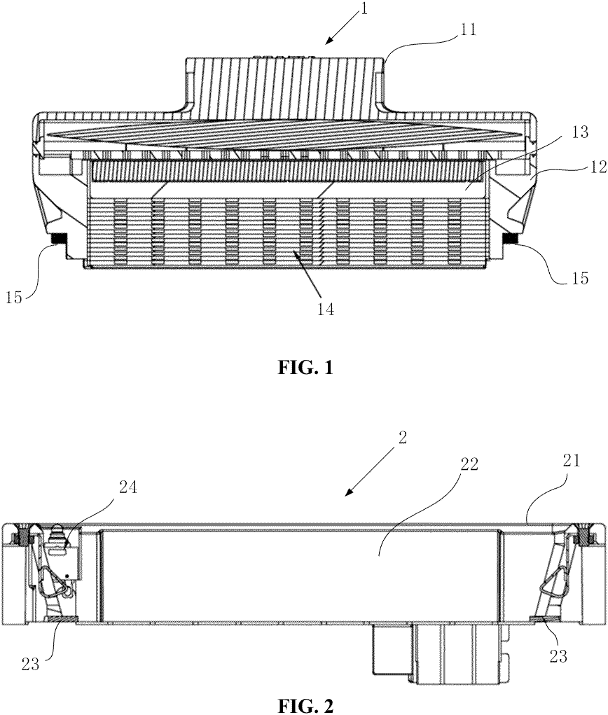

FIG. 1 is a schematic structural diagram of a replacement box 1 for a urinalysis device of the present disclosure. The replacement box for the urinalysis device comprises a box body upper cover 11 and a box body lower cover 12. The box body upper cover 11 and the box body lower cover 12 are connected to form a box body cavity 13. Test paper 14 is contained in the box body cavity 13. A bottom portion of the box body lower cover 12 is provided with a first magnetic attraction device 15.

Specifically, the replacement box 1 is replaced in the urinalysis device as a whole. The box body upper cover 11 and the box body lower cover 12 are connected to form the box body cavity 13 for containing the test paper 14. In an embodiment, the test paper 14 may be placed in the box body lower cover 12. Finally, the first magnetic attraction device 15 is installed on the bottom portion of the box body lower cover 12.

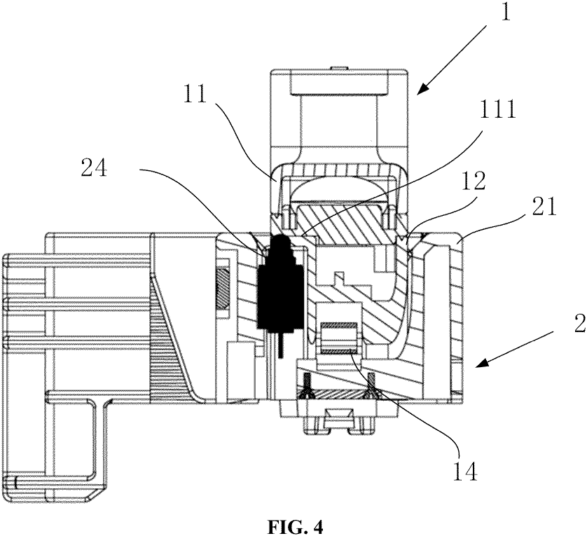

FIG. 4 is a sectional view of a replacement box mounting structure for the urinalysis device according to an example of the present disclosure. As shown in FIG. 4, when in use, the replacement box 1 is inserted into a replacement box bottom support 2 for the urinalysis device. A bottom portion of the replacement box bottom support 2 is provided with a second magnetic attraction device 23. The first magnetic attraction device 15 and the second magnetic attraction device 23 are attracted to each other, so that the replacement box 1 can be guided to reach a preset position.

According to the present disclosure, the replacement box is guided and positioned to the preset position through the first magnetic attraction device and the second magnetic attraction device. An in-place detection regarding whether the replacement box reaches the preset position is conducted through a detection switch. Thus, a guiding effect is provided for a user to mount or install the replacement box, and the in-place detection precision can be improved.

FIG. 1 shows a replacement box 1 for a urinalysis device in an embodiment of the present disclosure, The replacement box for the urinalysis device comprises a box body upper cover 11 and a box body lower cover 12. The box body upper cover 11 and the box body lower cover 12 are connected to form a box body cavity 13, test paper 14 is contained or housed in the box body cavity 13. A bottom portion of the box body lower cover 12 is provided with a first magnetic attraction device 15. The first magnetic attraction device 15 is a magnet or a magnetic attraction iron sheet. An upper cover bottom surface 111 of the box body upper cover 11 protrudes beyond the box body lower cover 12.

Specifically, the first magnetic attraction device 15 may be the magnet or the magnetic attraction iron sheet. When the first magnetic attraction device 15 is a magnet, a second magnetic attraction device 23 is a magnetic attraction iron sheet. When the first magnetic attraction device 15 is a magnetic attraction iron sheet, the second magnetic attraction device 23 is a magnet. The magnet may be replaced by any magnetic materials. The magnetic attraction iron sheet may be replaced by any materials that may be magnetically attracted to the magnet. The first magnetic attraction device 15 and the second magnetic attraction device 23 are attracted to each other, so that the replacement box 1 can be guided to reach a preset position.

As shown in FIG. 4, the upper cover bottom surface 111 of the box body upper cover 11 protrudes beyond the box body lower cover 12. When the replacement box 1 is inserted into a replacement box bottom support 2, the box body lower cover 12 is inserted into a bottom support cavity 22, and a protruding part or portion of the upper cover bottom surface 111 of the box body upper cover 11 may trigger a detection switch 24 of the replacement box bottom support 2, e.g., by pressing the detection switch 24, so that an in-place signal can be generated.

In the embodiment, the attraction is realized by using the magnet or the magnetic attraction iron sheet, the replacement box is guided and positioned to a preset position, and the detection switch is triggered by the box body upper cover to generate the in-place signal, so that an in-place detection precision can be improved.

FIG. 2 is a schematic structural diagram of a replacement box bottom support 2 for a urinalysis device of the present disclosure. The replacement box bottom support for the urinalysis device comprises a bottom support body 21. A bottom support cavity 22 for containing a replacement box is arranged in the bottom support body 21, a bottom portion of the bottom support cavity 22 is provided with a second magnetic attraction device 23, and a detection switch 24 is also fixed on the bottom support body 21.

Specifically, the replacement box bottom support 2 is used for containing the replacement box 1. The bottom support cavity 22 is arranged in the bottom support body 21, and the replacement box 1 is inserted into the bottom support cavity 22. The bottom portion of the bottom support cavity 22 is provided with the second magnetic attraction device 23.

As shown in FIG. 4, when in use, the replacement box 1 is inserted into the bottom support cavity 22. A bottom portion of the replacement box 1 is provided with a first magnetic attraction device 15. The first magnetic attraction device 15 and the second magnetic attraction device 23 are attracted to each other, so that the replacement box 1 can be guided to reach a preset position.

According to the present disclosure, the replacement box is guided and positioned to the preset position through the first magnetic attraction device and the second magnetic attraction device. An in-place detection regarding whether the replacement box reaches the preset position is conducted through the detection switch. Thus, a guiding effect is provided for a user to mount or install the replacement box, and the in-place detection precision can be improved.

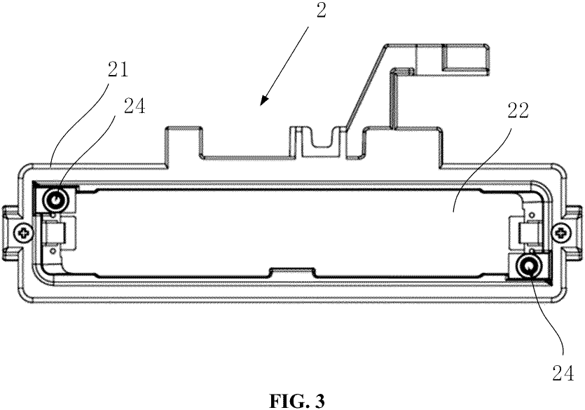

FIG. 2 and FIG. 3 show a replacement box bottom support 2 for a urinalysis device in an embodiment of the present disclosure. The replacement box bottom support for the urinalysis device comprises a bottom support body 21. A bottom support cavity 22 for containing a replacement box is arranged in the bottom support body 21. A bottom portion of the bottom support cavity 22 is provided with a second magnetic attraction device 23. A detection switch 24 and an indicating device in communication connection with the detection switch 24 are also fixed on the bottom support body 21. The second magnetic attraction device 23 is a magnetic attraction iron sheet or a magnet.

Specifically, a first magnetic attraction device 15 may be a magnet or a magnetic attraction iron sheet. When the first magnetic attraction device 15 is a magnet, the second magnetic attraction device 23 is a magnetic attraction iron sheet. When the first magnetic attraction device 15 is a magnetic attraction iron sheet, the second magnetic attraction device 23 is a magnet. The magnet may be replaced by any magnetic materials. The magnetic attraction iron sheet may be replaced by any materials that may be magnetically attracted to the magnet. The first magnetic attraction device 15 and the second magnetic attraction device 23 are attracted to each other, so that the replacement box 1 is guided to reach a preset position.

In an embodiment, two detection switches 24 may be respectively mounted on two sides of the bottom support body 21. The detection switch 24 may be a push switch, especially a high-precision switch. The detection switch 24 is in a free release state when there is no pressure applied to the detection switch 24. When the replacement box 1 is inserted into the replacement box bottom support 2, a box body lower cover 12 is inserted into the bottom support cavity 22, and a protruding part or portion of an upper cover bottom surface 111 of a box body upper cover 11 may trigger the detection switch 24 of the replacement box bottom support 2, e.g., by pressing the detection switch 24, so that an in-place signal can be generated.

The indicating device may be an indicator light, the indicating device is communicably coupled to the detection switch 24, and the indicating device, e.g., the indicator light, is turned on according to the in-place signal of the detection switch 24, so as to indicate that an in-place mounting is achieved, i.e., the replacement box 1 reaches the preset position.

In the embodiment, the attraction is realized by using the magnet or the magnetic attraction iron sheet, such that the replacement box can be guided and positioned to the preset position. Also, the in-place signal is generated through the detection switch, and whether in-place mounting is realized is indicated to a customer through the indicating device.

FIG. 4 is a sectional view of a replacement box mounting structure for a urinalysis device of the present disclosure. The replacement box mounting structure for the urinalysis device comprises the replacement box 1 as described above and the replacement box bottom support 2 as described above. The replacement box 1 is inserted into the replacement box bottom support 2 along a first direction. The first magnetic attraction device 15 and the second magnetic attraction device 23 are at least partially overlapped along the first direction when the replacement box 1 is inserted into the replacement box bottom support 2.

Specifically, when in use, the replacement box 1 is inserted into the replacement box bottom support 2 for the urinalysis device, the bottom portion of the replacement box 1 is provided with the first magnetic attraction device 15, and a bottom portion of the replacement box bottom support 2 is provided with the second magnetic attraction device 23. The first magnetic attraction device 15 and the second magnetic attraction device 23 are at least partially overlapped along the first direction, so that the first magnetic attraction device 15 and the second magnetic attraction device 23 can be attracted to each other and the replacement box 1 can be guided to reach a preset position.

According to the present disclosure, the replacement box is guided and positioned to the preset position through the first magnetic attraction device and the second magnetic attraction device. An in-place detection regarding whether the replacement box reaches the preset position is conducted through the detection switch. Thus, a guiding effect is provided for a user to mount or install the replacement box, and the in-place detection precision is improved.

FIG. 4 is a sectional view of a replacement box mounting structure for a urinalysis device of the present disclosure. The replacement box mounting structure for the urinalysis device comprises the replacement box 1 as described above and the replacement box bottom support 2 as described above. The replacement box 1 is inserted into the replacement box bottom support 2 along a first direction. The first magnetic attraction device 15 and the second magnetic attraction device 23 are at least partially overlapped along the first direction when the replacement box 1 is inserted into the replacement box bottom support 2.

The first magnetic attraction device 15 is a magnet and the second magnetic attraction device 23 is a magnetic attraction iron sheet; or the first magnetic attraction device 15 is a magnetic attraction iron sheet and the second magnetic attraction device 23 is a magnet. The magnet may be replaced by any magnetic materials. The magnetic attraction iron sheet may be replaced by any materials that may be magnetically attracted to the magnet.

A height from the upper cover bottom surface 111 of the box body upper cover 11 protruding beyond the box body lower cover 12 to a bottom portion of the first magnetic attraction device 15 is smaller than a height from a top portion of the second magnetic attraction device 23 to a top portion of the detection switch 24. When the box body lower cover 12 is inserted into the bottom support cavity 22, the box body upper cover 11 presses the detection switch 24.

Specifically, the detection switch 24 may be a push switch. The height from the upper cover bottom surface 111 of the box body upper cover 11 protruding beyond the box body lower cover 12 to the bottom portion of the first magnetic attraction device 15 is smaller than the height from the top portion of the second magnetic attraction device 23 to the top portion of the detection switch 24. When the replacement box 1 is inserted into the replacement box bottom support 2, the box body lower cover 12 is inserted into the bottom support cavity 22, so that a protruding part of the upper cover bottom surface 111 of the box body upper cover 11 can trigger the detection switch 24 of the replacement box bottom support 2, e.g., by pressing the detection switch 24, to generate an in-place signal.

In the embodiment, the attraction is realized by using the magnet or the magnetic attraction iron sheet, so that the replacement box can be guided and positioned to a preset position. Also, the detection switch is triggered by the box body upper cover to generate the in-place signal, so that an in-place detection precision can be improved.

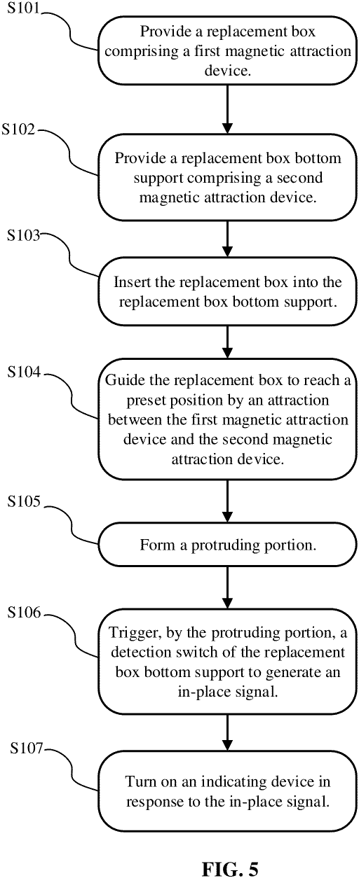

FIG. 5 is a flow chart of a method for assembling a replacement box mounting structure for a urinalysis device according to an example of the present disclosure. The replacement box mounting structure to be assembled by the method may be the replacement box mounting structure according to any of the foregoing embodiments. The replacement box and the replacement box bottom support of the replacement box mounting structure are described above in the present disclosure. The replacement box, the replacement box bottom support, and the replacement box mounting structure are configured to perform an operation, function, or the like as described above in the present disclosure.

At act S101, a user may provide the replacement box 1 comprising the first magnetic attraction device 15. The first magnetic attraction device 15 is disposed at the bottom portion of the box body lower cover 12 of the replacement box 1.

At act S102, the user may provide the replacement box bottom support 2 comprising the second magnetic attraction device. The second magnetic attraction device 23 is disposed at the bottom portion of the bottom support cavity 22.

At act S103, the user may insert the replacement box 1 into the bottom support cavity 22 of the replacement box bottom support 2. The direction along which the replacement box 1 into the bottom support cavity 22 refer to the “first direction” in the present disclosure.

At act S104, an attraction between the first magnetic attraction device 15 and the second magnetic attraction device 23 may guide the replacement box 1 to reach a preset position. As described above, when the first magnetic attraction device 15 is a magnet, the second magnetic attraction device 23 is a magnetic attraction iron sheet. When the first magnetic attraction device 15 is a magnetic attraction iron sheet, the second magnetic attraction device 23 is a magnet. The magnet may be replaced by any magnetic materials. The magnetic attraction iron sheet may be replaced by any materials that may be magnetically attracted to the magnet. The first magnetic attraction device 15 and the second magnetic attraction device 23 are attracted to each other, so that the replacement box 1 can be guided to reach the preset position.

At act S105, the user may form the protruding portion by allowing the upper cover bottom surface 111 of the box body upper cover 11 of the replacement box 1 to protrude beyond the box body lower cover 12 of the replacement box 1.

At act S106, the protruding portion may trigger the detection switch 24 of the replacement box bottom support 2 to generate the in-place signal. Specifically, when the replacement box 1 reaches the preset position, the protruding portion may press the detection switch 24 and thus trigger the detection switch 24 to generate the in-place signal.

In another embodiment, two detection switches 24 may be respectively disposed on two sides of the bottom support body 21. When the replacement box 1 is inserted into the bottom support cavity 22 of the replacement box bottom support 2, the in-place signal may be generated when the two detection switches 24 are both triggered by the protruding portion.

At act S107, the in-place signal generated by the detection switch 24 may turn on the indicating device. The indicating device is communicably coupled to the at least one detection switch. As described above, the indicating device may be an indicator light configured to indicate that an in-place mounting is achieved, i.e., the replacement box 1 reaches the preset position.

The above embodiments merely express several embodiments of the present disclosure, and the descriptions thereof are more specific and detailed. However, it should not be understood as a limitation to the patent scope of the present disclosure. It should be noted that those of ordinary skill in the art may make a plurality of variations and improvements without departing from the conception of the present disclosure. These variations and improvements shall all fall within the protection scope of the present disclosure. Therefore, the protection scope of the patent according to the present disclosure shall be subjected to the appended claims.

1-10. (canceled)

11. A replacement box for a urinalysis device, the replacement comprising:
a box body upper cover;
a box body lower cover configured to be connected to the box body upper cover to form a box body cavity, the box body cavity configured to house test paper; and
a first magnetic attraction device disposed at a bottom portion of the box body lower cover,
wherein the first magnetic attraction device is configured to be attracted to guide the replacement box to reach a preset position when the replacement box is inserted into a replacement box bottom support.

12. The replacement box according to claim 11, wherein the first magnetic attraction device is configured to be attracted to a second magnetic attraction device of the replacement box bottom support so as to reach the preset position when the replacement box is inserted into the replacement box bottom support.

13. The replacement box according to claim 12, wherein the first magnetic attraction device is made of a magnetic material or a metal material.

14. The replacement box according to claim 13, wherein one of the first magnetic attraction device and the second magnetic attraction device is made of the magnetic material and the other of the first magnetic attraction device and the second magnetic attraction device is made of the metal material.

15. The replacement box according to claim 11, wherein an upper cover bottom surface of the box body upper cover protrudes beyond the box body lower cover to form a protruding portion.

16. The replacement box according to claim 15, wherein the protruding portion of the box body upper cover is configured to trigger at least one detection switch of the replacement box bottom support so as to generate an in-place signal by pressing the at least one detection switch.

17. The replacement box according to claim 16, wherein the in-place signal is configured to turn on an indicating device of the replacement box bottom support, and
wherein the indicating device is communicably coupled to the at least one detection switch.

18. The replacement box according to claim 16, wherein the protruding portion of the box body upper cover is configured to trigger two detection switches of the replacement box bottom support, and
wherein the two detection switches are respectively disposed on two sides of the replacement box bottom support.

19. A replacement box bottom support for a urinalysis device, the replacement box bottom support comprising:
a bottom support body;
a bottom support cavity disposed in the bottom support body and configured to receive a replacement box; and
a magnetic attraction device disposed at a bottom portion of the bottom support cavity,
wherein the magnetic attraction device of the replacement box bottom support is configured to guide the replacement box to reach a preset position when the replacement box is inserted into the replacement box bottom support.

20. The replacement box bottom support according to claim 19, wherein the magnetic attraction device of the replacement box bottom support is configured to be attracted to a magnetic attraction device of the replacement box so as to guide the replacement box to reach the preset position when the replacement box is inserted into the replacement box bottom support.

21. The replacement box bottom support according to claim 20, wherein the magnetic attraction device of the replacement box bottom support is made of a metal material or a magnetic material.

22. The replacement box bottom support according to claim 21, wherein one of the magnetic attraction device of the replacement box and the magnetic attraction device of the replacement box bottom support is made of the magnetic material and the other of the magnetic attraction device of the replacement box and the magnetic attraction device of the replacement box bottom support is made of the metal material.

23. The replacement box bottom support according to claim 19, further comprising at least one detection switch disposed at the bottom support body and configured to detect whether the replacement box reaches the preset position.

24. The replacement box bottom support according to claim 23, wherein the at least one detection switch is triggered by a protruding portion of the replacement box to generate an in-place signal, and
wherein an upper cover bottom surface of a box body upper cover of the replacement box protrudes beyond a box body lower cover of the replacement box to form the protruding portion.

25. The replacement box bottom support according to claim 24, further comprising an indicating device communicably coupled to the at least one detection switch,
wherein the indicating device is turned on in response to the in-place signal generated by the detection switch.

26. The replacement box bottom support according to claim 24, wherein a height from the upper cover bottom surface of the box body upper cover protruding beyond the box body lower cover to a bottom portion of the magnetic attraction device of the replacement box is smaller than a height from a top portion of the magnetic attraction device of the replacement box bottom support to a top portion of the at least one detection switch.

27. The replacement box bottom support according to claim 23, wherein the at least one detection switch comprises two detection switches respectively disposed on two sides of the replacement box bottom support.

28. A method for assembling a replacement box mounting structure for a urinalysis device, the method comprising:
providing a replacement box comprising a first magnetic attraction device disposed at a bottom portion of a box body lower cover of the replacement box;
providing a replacement box bottom support comprising a second magnetic attraction device disposed at a bottom portion of a bottom support cavity;
inserting the replacement box into the bottom support cavity of the replacement box bottom support; and
guiding the replacement box to reach a preset position by an attraction between the first magnetic attraction device and the second magnetic attraction device.

29. The method according to claim 28, further comprising:
forming a protruding portion by allowing an upper cover bottom surface of a box body upper cover of the replacement box to protrude beyond the box body lower cover of the replacement box; and
triggering, by the protruding portion, at least one detection switch of the replacement box bottom support to generate an in-place signal.

30. The method according to claim 29, further comprising:
turning on an indicating device, communicably coupled to the at least one detection switch, in response to the in-place signal generated by the at least one detection switch.
